(19) United States
(12) Patent Application Publication
(10) Pub. No.: US 2021/0059520 A1
(43) Pub. Date: Mar. 4, 2021

(54) OPHTHALMIC SYSTEMS AND METHODS FOR DIRECT RETINAL STIMULI WITH LOCAL RETINAL ANGLE OF INCIDENCE CONTROL

(71) Applicant: Johnson & Johnson Vision Care, Inc., Jacksonville, FL (US)

(72) Inventor: Derek Nankivil, Jacksonville, FL (US)

(21) Appl. No.: 17/011,275

(22) Filed: Sep. 3, 2020

Related U.S. Application Data
(60) Provisional application No. 62/895,273, filed on Sep. 3, 2019.

(51) Int. Cl. A61B 3/10; G01B 9/02

(52) U.S. Cl. CPC A61B 3/102; G01B 9/02004; G01B 9/02063

(57) ABSTRACT

Ophthalmic systems and methods for direct retinal stimuli and OCT retinal imaging with local retinal angle of incidence control are disclosed. According to an aspect, an ophthalmic system includes an optotype generator configured to provide a direct optical stimulus to a retina of an eye, wherein an angle of incidence of the direct optical stimulus upon the retina may be adjusted. The imaging system also includes an optical coherence tomography (OCT) imaging system configured to generate OCT images of the retina of the eye used to characterize the response to the direct optical stimulus.

### I. CROSS-REFERENCE TO RELATED APPLICATIONS

This patent application claims the benefit of United States Provisional Patent Application No. 62/895,273 filed Sep. 3, 2019. The contents of each are herein incorporated by reference.

### II. BACKGROUND OF THE INVENTION

### a. Field of the Invention

The described embodiments relate generally to ophthalmic medical devices. More particularly, the embodiments relate to an ophthalmic system capable of presenting direct retinal stimuli with local retinal angle of incidence control while simultaneously acquiring optical coherence tomography (OCT) volumes of the retina.

### b. Background and Discussion of the Related Art

With the rapidly growing incidence of myopia around the world, it is anticipated that the concomitant sight-threatening retinal complications will continue to increase at an alarming pace. Of great concern for myopic individuals is the condition's potential progression to high myopia, for example greater than five or six diopters (that is, according to sign convention, <−5.00 or −6.00 D), which dramatically affects one's ability to function without optical aids. High myopia is also associated with an increased risk of retinal disease, cataracts, and glaucoma, among other things.

Historically, corrective lenses were used to alter the gross focus of the eye to render a clearer image at the retinal fovea, by shifting the focus from in front of the retina to correct myopia, or from behind the retina to correct hyperopia, respectively. However, the corrective approach to the conditions does not address the cause of the condition but rather merely addresses the symptoms. Given the scale and economic ramifications of this epidemic, efforts to mitigate or prevent myopia progression have accelerated recently. Myopia typically occurs due to excessive axial growth or elongation of the eye. It is now generally understood that axial eye growth can be influenced by the quality and focus of the retinal image. Experiments have illustrated that altering retinal image quality can lead to consistent and predictable changes in eye growth. Furthermore, defocusing the retinal image through positive lenses (myopic defocus) or negative lenses (hyperopic defocus), is known to lead to predictable (in terms of both direction and magnitude) changes in eye growth, consistent with the eyes growing to compensate for the imposed defocus.

The changes in eye length associated with optical blur have been shown to be modulated by changes in both scleral growth and choroidal thickness. Blur with positive lenses, which leads to myopic blur, thickening of the choroid, and decrease in scleral growth rate, results in reduced axial growth rate. Blur with negative lenses, which leads to hyperopic blur, thinning of the choroid, and increase in scleral growth rate, results in increased axial growth rate. These eye growth changes in response to retinal image defocus have been demonstrated to be largely mediated through local retinal mechanisms, as eye length changes still occur when the optic nerve is damaged, and imposing defocus on local retinal regions has been shown to result in altered eye growth localized to that specific retinal region.

In humans, there is both indirect and direct evidence that supports the notion that retinal image quality can influence eye growth. A variety of different ocular conditions, all of which lead to a disruption in form vision, such as ptosis, congenital cataract, corneal opacity, vitreous hemorrhage and other ocular diseases, have been found to be associated with abnormal eye growth in young humans, which suggests that relatively large alterations in retinal image quality do influence eye growth in human subjects. The influence of more subtle retinal image changes on eye growth in humans has also been hypothesized based on optical errors in the human focusing system that may provide a stimulus for eye growth and myopia development in humans.

Evidence further suggests that the retina alone can detect the sign of defocus. More specifically, using high-resolution imaging capabilities provided by optical coherence tomography (OCT), researchers have shown that the choroid appears to have a role in the regulation of eye growth and may respond to defocus cues. Furthermore, models suggest that the retina may detect the sign of defocus by assessing leakage to the intra-cone spacing, which is a function of angle of incidence (AOI). Further modeling also suggests that the retina may detect the sign of defocus by assessing leakage to the intra-cone spacing, which is a function of AOI.

Ophthalmic lenses designed to slow, retard, or prevent myopia progression have been developed in appreciation of one or more aspects of this body of evidence, including those lens designs disclosed and claimed by U.S. Patent Publication No. 2019/0227342 (A1), entitled “Ophthalmic Lens With An Optically Non-Coaxial Zone for Myopia Control” to Brennan et al. (hereinafter “Brennan”). Among other things, some embodiments of the lens designs of Brennan utilize an annular treatment zone with a power profile that generates a focal ring which selectively modulates the AOI.

Further advancement in the design of ophthalmic devices to slow or prevent myopia progression (and potentially other conditions of the eye) would benefit from objective data on the response of the retina, and in particular the choroid, to AOI or the defocus of a stimulus in human subjects in vivo. Ophthalmic diagnostic systems such as OCT typically employ simple cross-like stimuli that are foveated to permit subject fixation and mitigate motion artifacts. However, commercial OCT systems do not provide a means to control these aspects of the stimuli such as wavelength, intensity, polarization and angle of incidence.

Accordingly, there remains a need for effective systems and methods capable of controlling these aspects and obtaining data capable of characterizing their effect on the eye. Furthermore, there remains a need to apply such findings to enable early diagnosis of retinal or neurological disease and an improved sensitivity to therapeutic efficacy needed to enhance approaches to individualized medicine; and to develop improved ophthalmic devices capable of slowing or prevent conditions of the eye including but not limited to myopia. Such ophthalmic systems and methods could be used to characterize the relationship between parameters such as stimuli defocus, wavelength, intensity, polarization and angle of incidence and anatomical structural characteristics of the retina in health and disease.

### III. SUMMARY OF THE INVENTION

Disclosed herein are ophthalmic systems and methods capable of presenting direct retinal stimuli with local retinal angle of incidence control while simultaneously acquiring optical coherence tomography volumes of the retina.

In an embodiment, an ophthalmic system includes an optotype generator configured to provide a direct optical stimulus to a retina of an eye, wherein an angle of incidence of the direct optical stimulus upon the retina may be adjusted; and an OCT imaging system configured to generate OCT images of the retina of the eye in response to the direct optical stimulus.

In a further embodiment, a method includes providing, via an optotype generator, a direct optical stimulus to a retina of an eye; selectively adjusting the angle of incidence of the direct optical stimulus upon the retina; and generating, via an optical coherence tomography (OCT) imaging system, a plurality of OCT images of the retina of the eye in response to the direct optical stimulus.

### IV. BRIEF DESCRIPTION OF THE DRAWINGS

The foregoing and other features and advantages of the invention will be apparent from the following, more particular description of preferred embodiments of the invention, as illustrated in the accompanying drawings.

FIG. 1 shows a representative schematic of an ophthalmic system including an optotype generator and OCT imaging system according to an embodiment.

FIG. 2 shows a flow diagram for a method of providing stimulation to a retina and imaging the response of the retina in accordance with an embodiment.

FIG. 3 shows representative schematic of ophthalmic system including an optotype generator incorporating lasers and an OCT imaging system according to an embodiment.

FIG. 4 shows a representative schematic of ophthalmic system including an optotype generator incorporating a video screen and an OCT imaging system according to an embodiment.

FIG. 5 shows a ray diagram of an OCT imaging system according to an embodiment.

FIG. 6 shows a ray diagram of an optotype generator according to an embodiment.

### V. DETAILED DESCRIPTION

Reference will now be made in detail to representative embodiments illustrated in the accompanying drawings wherein reference numerals indicate certain elements. The following descriptions are not intended to limit the myriad embodiments to one preferred embodiment. To the contrary, it is intended to cover alternatives, modifications, and equivalents as can be included within the spirit and scope of the described embodiments as defined by the appended claims.

References to “one embodiment,” “an embodiment,” “some embodiments,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

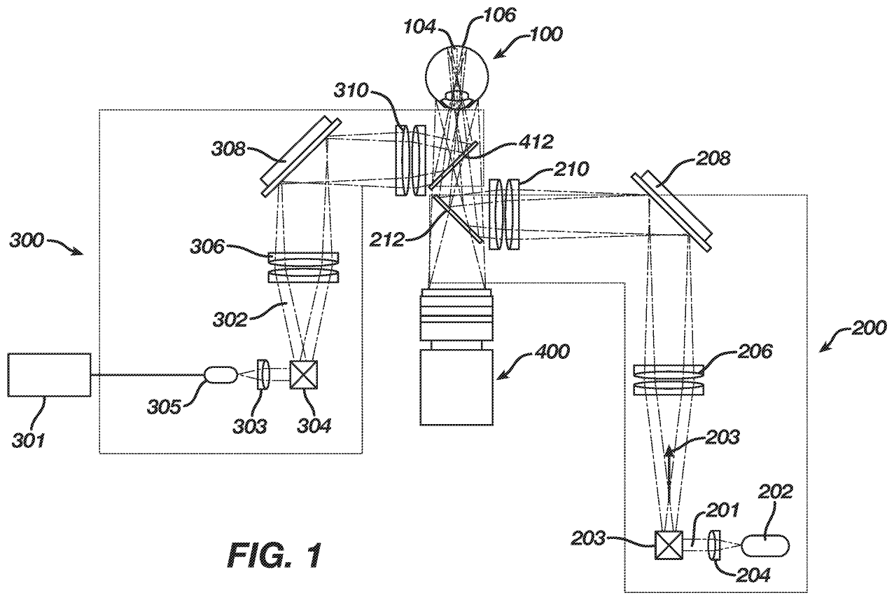

FIG. 1 illustrates a representative schematic of an ophthalmic system including an optotype generator 200 and OCT imaging system 300, sometimes referred to as an OCT sample arm, according to an embodiment. A subject eye 100, including a retina 104 and choroid 106, is positioned in optical line of sight of light beams associated with an optotype generator 200, an OCT imaging system 300, and a camera 400. Optotype generator 200 is configured to provide a direct optical stimulus to a retina of eye 100, and in this embodiment retina 104 and choroid 106 in particular. A direct optical stimulus may take numerous forms but may be for example a light beam 201 projected directly upon the retina 104 via an optical path to produce a stimulation pattern upon the retina. As discussed in embodiments herein, and as understood by one of skill in the art, direct optical stimuli may also include a laser stimulus or stimulus from a video screen, digital micromirror device (DMD), or other digital projection sources. One skilled in the art will also appreciate that, in addition to the particular topologies and embodiments specifically outlined herein, an object-side telecentric configuration could be employed to enable improved light transfer. Optotype generator 200 may be configured such that the angle of incidence of the direct optical stimulus upon the retina may be adjusted within a desired range, which may include but is not limited to about ±14° at the fovea. Adjustment of retinal AOI may be achieved using an integrated pupil tracking system that permits closed loop control of the entry pupil position. One suitable pupil tracking system is described in “Pupil tracking Optical Coherence Tomography for Precise Control of Pupil Entry Position” Biomedical Optics Express, Carrasco-Zevallos O, Nankivil D, Keller B, Viehland C, Lujan J, Izatt J A., 2015 Sep. 1; 6(9):3405-19.

The optical stimulus produced by optotype generator 200 may originate from a light source 202, which may be composed of one or more light emitting diodes (LEDs). While not illustrated, light source 202 may be operably connected to and controlled by OCT engine 301. Light source 202 may produce primary colors and may be implemented as an at least three-channel primary color (e.g., red, green, blue or other desired colors) array. In some cases, a selectively controllable shutter may be configured to control the projection of light among primary colors. Or, alternatively, a direct light source may be paired with one or more filters selected to produce primary colors via secondary filtration of the direct light source 202. By way of example, color could be controlled through selective shuttering of individual primaries with any of the aforementioned approaches, direct electrical control of the individual sources, or through secondary filtration with a Lyot, dichroic, or absorptive filter. A lens 204 may be positioned downstream from light source and configured to pass the generated light along a direction indicated by arrow 203. A retinal scanner 203 may be included for retinal scanning and may be controlled to produce the desired stimuli. Lens pair 206 may be positioned downstream from retinal scanner 203 along beam path 203. Moving further along lens path 203, a pupil scanner 208 may receive and redirect light toward lens pair 210. Pupil scanner 208 may be controllable to pivot in real-time along multiple axes to compensate for eye motion and thus maintain the desired entry pupil position and AOI. A dichroic mirror 212 may be positioned to receive the light from lens 210 and redirect the light toward the eye 100.

In some embodiments, optotype generator 200 may be configured to modulate a frequency or intensity of the direct optical stimulus. Modulation of the frequency or intensity of the direct optical stimulus may be achieved through various means, including but not limited to a shutter, chopper, acousto-optic, or an electro-optic modulator. Furthermore, optotype generator 200 may include an electrically controllable birefringent element, such as a polarizer, waveplate, or the like to control the polarization state or intensity of the stimuli. Further still, optotype generator 200 may include be configured to facilitate adjustment of a focus of the direct optical stimulus such as by the inclusion of a Badal optometer, tunable lens, variable phase mask, adjustable imaging telescopes, e.g, 4F telescope, or the like.

The ophthalmic system further includes an OCT imaging system 300 configured to generate OCT images of the retina of the eye in response to the optical stimulus of optotype generator 200. An OCT engine 301 in this embodiment may be a swept source OCT engine which may utilize a fiber-based interferometer topology, such as e.g., a Michelson, Mach-Zehnder, transmissive or spectrally balanced interferometer, although other OCT engines and interferometers may be possible. As used herein, an OCT engine refers to a computer processing system including at least a broadband light source, interferometer and processor and a computer readable storage medium having instructions stored therein that, when executed by the processor, cause the OCT imaging system 300 to function as described herein. In this embodiment, the OCT imaging system 300 is capable of imaging the retina over a 60-degree field of view (FOV) and comprises a swept-frequency laser centered at about 1060 nm with a sweep rate of about 200 kHz and a bandwidth of about 100 nm. Further still, OCT imaging system 300 may include a Badal optometer configured to facilitate adjustment of a focus of the sample arm beam to compensate for refractive error of eye.

Referring still to FIG. 1, light beam 302 originating from OCT engine 301 passes through lens 303 before entering retinal scanner 304. In the same manner described above with respect to optotype generator 200, pupil scanner 308 may be configured for real-time tracking and compensation of the pupil's movement. Light beam 302 further travels through lens pair 306 to pupil scanner 308 before being redirected through lens pair 310. Finally, light beam 302 reflects off dichroic mirror 412 before entering eye 100.

The Ophthalmic system further includes a camera 400 positioned and configured to capture images of the eye 104. This sequence of images may, in some embodiments, be captured as or assembled into video of the eye. By capturing these images and video, pupil motion is characterized and used as input to the pupil tracking systems.

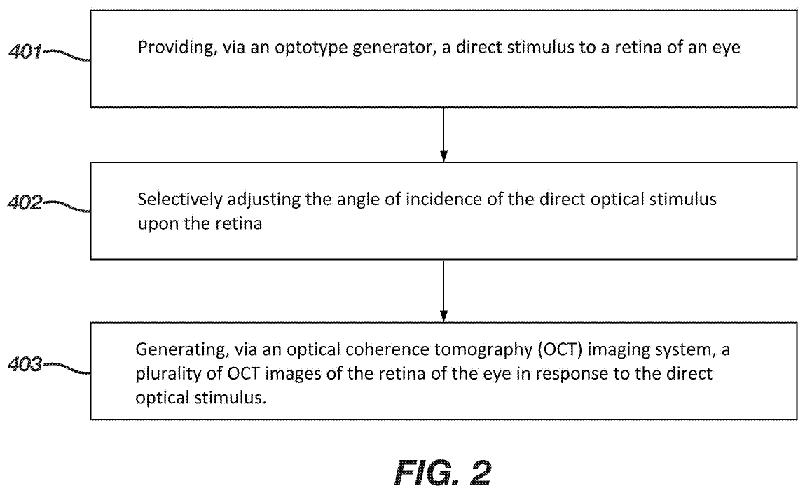

FIG. 2 shows a flow chart of an exemplary method of providing direct retinal stimulation to a retina and imaging the response of the retina in accordance with an embodiment. The example method may be implemented by the ophthalmic system of FIG. 1. However, it should be understood that the method may be implemented by any suitable ophthalmic imaging system having a means of simultaneously imaging and stimulating the retina. It is also noted that the steps of the method may be implemented repeatedly or in a recursive fashion or in an order different than illustrated.

Step 401 includes providing, via an optotype generator, a direct optical stimulus to a retina of an eye. Step 402 includes selectively adjusting the angle of incidence of the direct optical stimulus upon the retina. And step 403 includes generating, via an optical coherence tomography (OCT) imaging system, a plurality of OCT images of the retina of the eye and measures of choroidal thickness which may change in response to the direct optical stimulus.

Understanding that the exemplary method of FIG. 2 may be performed with the ophthalmic system described with reference to FIG. 1, it is further understood that the details and variations discussed with reference to the system apply equally to the performance of the method. Furthermore, the direct optical stimulus produced by the optotype generator may comprise at least three channels wherein each channel is composed of a different color that may serve as primaries. Further still, the method may involve selectively controlling a shutter or other mechanism to control a projection of light among an individual or plurality of primary colors to produce a stimulus of any color. The method may involve controlling, via an integrated pupil tracking system, an entry position to the pupil of the direct optical stimulus; modulating a frequency or intensity of the direct optical stimulus, wherein the frequency or intensity of the direct optical stimulus may be modulated via at least one of a chopper, acoustic optic, or an electro optic modulator.

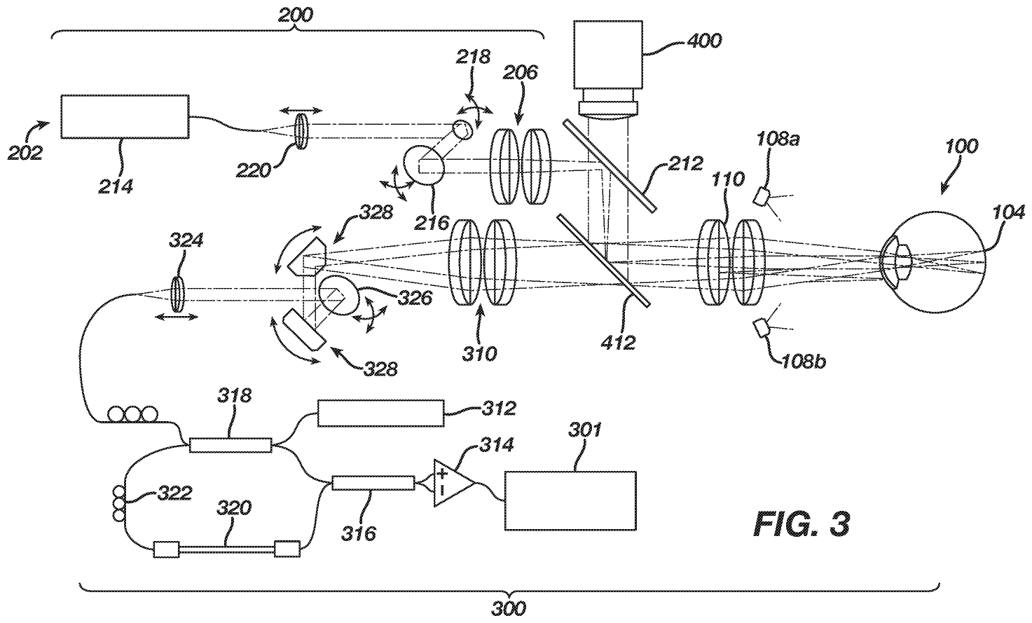

Turning to FIG. 3, a representative schematic of ophthalmic system including an optotype generator 200 incorporating lasers 214 and an OCT imaging system 300 according to an embodiment is illustrated. In particular, an optotype generator 200 is implemented utilizing three lasers 214 at visible wavelengths integrated into a single source 202. Optotype generator 200 in this embodiment includes three optotype light sources 214, which in this exemplary implementation are lasers selected at visible wavelengths integrated into a single source 202. The wavelengths of these lasers may be within the ranges of 400-700 nm and in particular may be 473 nm (blue), 532 nm (green), and 640 nm (red). The lasers in this embodiment may be integrated to act as a single source using a wavelength division multiplexer (such as Thorlabs RBB Combiner RGB26HF-RGB Combiner) or in free space with a series of corresponding dichroic mirrors or filters. Alternatively, or additionally, laser(s) may also include a supercontinuum laser operating at or filtered to the above wavelengths. Optotype diopter focus control 220 may be provided using a lens on a mechanical stage, a tunable lens, or a deformable mirror although other focusing methods may be possible. Here, a diopter focus control 220 utilizes a lens on a linear translation mount following the 3-laser source fiber to allow for focusing and defocusing of an optical stimulus (also referred to interchangeably as visual stimulus herein) at the retinal image plane. A pupil scanner, referred to interchangeably in some embodiments as optotype 2d motion compensating mirror 218, is placed offset from a retinal scanner, referred to interchangeably in some embodiments as an the optotype 2D position scanning mirror 216, to compensate for patient motion and to allow programmatic control over the offset of entrance of the optical stimulus to the ocular pupil. In some embodiments, this control could be used to enable local control of the angle of incidence across the retina. Optotype visual stimulus patterns may be generated through the use of a 2D position scanning mirror 216. Other embodiments may utilize alternative 2D scanning mechanisms, such as a galvo pair, conjugate galvo pair, spatial light modulator, or resonate mirror (with or without a modulating light source). Optotype 2D motion compensating mirror 218 and optotype 2d position scanning mirror 216 are communicatively coupled to a processor (not pictured) and responsive to signals from the processor that drive the mirror positions in real time to compensate for patient motion, as detected by iris camera 400, and, in the case of the optotype 2d position scanning mirror 216, as desired to generate the desired visual stimulus pattern. A pair of lenses 206 or lens systems are configured in a 4F imaging telescope configuration such that the 2D position scanning mirror 216 is at an image conjugate of the ocular pupil 104 where the second lens system is the same second lens system 310 used in the OCT sample arm. Dichroic mirrors 212 are placed within the 4F imaging telescope to integrate optotype visual stimulus wavelengths with the iris camera 400 and OCT system 300.

OCT imaging system 300 includes an OCT engine 301, also referred to herein as an acquisition and processing computer, which may be a local, commercially available personal computer. The OCT source in this embodiment includes an external tunable cavity wavelength swept laser 312 from 980-1100 nm with a sweep rate of 200 kHz. Detection of OCT interferometric signal is performed with an indium gallium aresenide (InGaAs) high-speed balanced photo receiver 314 and digitized by a high-speed digitizer in the OCT engine 301 also referred to herein as an acquisition computer. An OCT interferometer consists of two 2×2 fusion spliced fiber couplers 318 and 316, one with an 80% to 20% coupling ratio and the other with a 50% to 50% coupling ratio, respectively. The OCT reference arm is in a transmissive topology such that light from one fiber output of the 80/20 coupler 318 is coupled into a fiber input of the 50/50 coupler 316. Polarization states between reference and sample arms are matched through the use of fiber paddle polarization controllers 322, such as Thorlabs FPC030. The output light is then collimated with a collimator 320, such as Thorlabs T06APC-1064. The other fiber output of the 80/20 coupler 318 is connected to the OCT sample arm. A tunable lens 324, which may be electronically or manually controlled, enables focusing to compensate for diopter variations between individuals. The tunable lens 324 may be electronically tunable, such as an Optotune EL-3-10, or manually focusable, such as an Optotune ML-20-37. An OCT 2D scanning motion compensating mirror 326 is placed offset from the offset galvo scanning mirrors 328 to compensate for motion during OCT image acquisition. Offset galvo scanning mirrors provide 2d scanning of the OCT beam to enable image formation. OCT 2D motion compensating mirror 326 and OCT offset galvo scanning mirrors 328 are communicatively coupled to a processor and responsive to signals from the processor that drive the mirror positions in real time to compensate for patient motion, as detected by iris camera 400, and, in the case of the OCT offset galvo scanning mirrors 328, as desired to generate the desired imaging scan pattern. A pair of lenses or lens systems 310 are configured in a 4F imaging telescope configuration such that the offset galvo scanning mirrors 328 are at an image conjugate of the ocular pupil and the second lens system is the second lens system of the optotype 4F imaging telescope. A dichroic mirror 412 is placed within the 4F imaging telescope to integrate optotype visual stimulus wavelengths. Finally, lens pair 110, here an N-FK51A Lens pair, functions as second lens pair of OCT imaging 4F telescope and second lens pair of optotype relay 4F telescope in the optical path to the retina 104 of eye 100. Iris camera illumination is provided by a ring of other configuration of LEDs 108a and 180b.

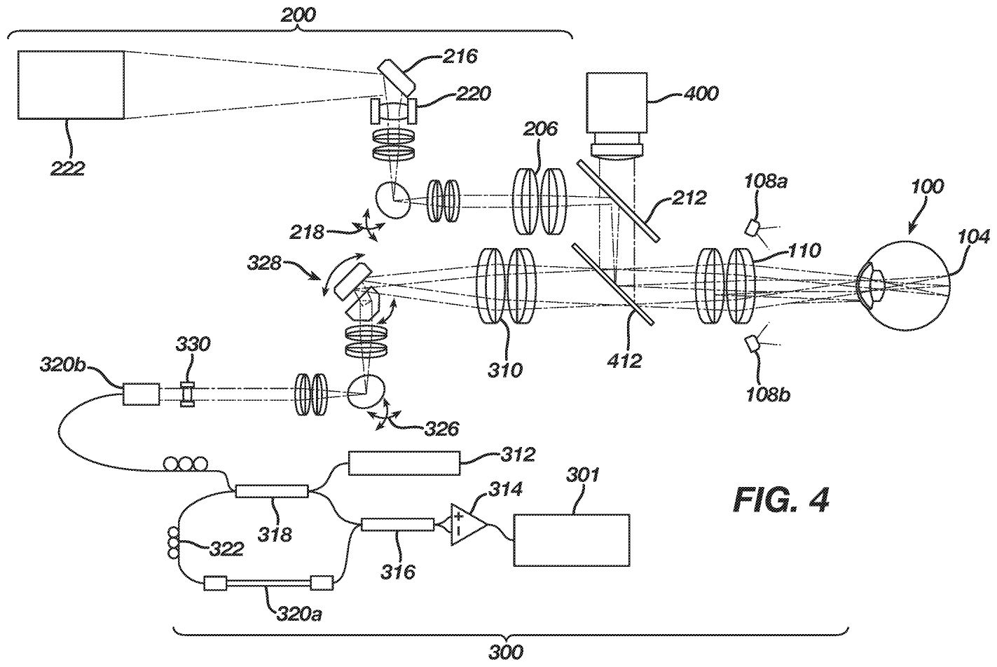

Referring now to FIG. 4, a representative schematic is provided for an ophthalmic system including an optotype generator 200 incorporating a video screen 222 and an OCT imaging system 300 according to an embodiment. In particular, optotype generator 200 produces visual stimuli in this embodiment by way of a video screen 222 with visible wavelengths (blue, green, and red). The video screen 222 may be an organic light emitting diode (OLED) display such as DFRobot DFR0524. Optotype diopter focus control 220 may be provided using a lens on a mechanical stage, a tunable lens, or a deformable mirror although other focusing methods will be apparent to those of skill in the art. Here, diopter focus control 220 utilizes a manual tunable lens a focal length away from the first lens system of a 2D motion compensating mirror 218 of a 4F telescope. An optotype 2d motion compensating mirror 218 is placed conjugate to an image plane of the retina 104 to compensate for patient motion and to allow programmatic control over the offset of entrance of the optical stimulus to the ocular pupil and is located at an intermediate focal plane between the two lens systems of the 2D motion compensating mirror 218 of a 4F telescope. This control may be used to enable local control of the angle of incidence across the retina 104. Optotype visual stimulus patterns may be generated through the use of a 2D position scanning mirror 218. Other embodiments may utilize alternative 2D scanning mechanisms, such as a galvo pair, conjugate galvo pair, spatial light modulator, or resonate mirror (with or without a modulating light source). Optotype 2D motion compensating mirror 218 and optotype 2d position scanning mirror 216 are communicatively coupled to a processor (not shown) and responsive to signals from the processor that drive the mirror positions in real time to compensate for patient motion, as detected by iris camera 400, and, in the case of the optotype 2d position scanning mirror 216, as desired to generate the desired visual stimulus pattern. A pair of lenses 206 or lens systems are configured in a 4F imaging telescope configuration such that the 2D position scanning mirror is at an image conjugate of the ocular pupil where the second lens system is the same second lens system used in the OCT sample arm. Dichroic mirrors 212 are placed within the 4F imaging telescope to integrate optotype visual stimulus wavelengths with the iris camera and OCT system.

OCT imaging system 300 includes an OCT engine 301, also referred to herein as an acquisition and processing computer, which may be a local, commercially available personal computer. The OCT source in this embodiment includes an external tunable cavity wavelength swept laser 312 from 980-1100 nm with a sweep rate of 200 kHz. Detection of OCT interferometric signal is performed with an InGaAs high-speed balanced photo receiver 314 and digitized by a high-speed digitizer in the acquisition computer 301. The OCT interferometer consists of two 2×2 fusion spliced fiber couplers 318 and 316, one with an 80% to 20% coupling ratio and the other with a 50% to 50% coupling ratio, respectively. The OCT reference arm is in a transmissive topology such that light from one fiber output of the 80/20 coupler 318 is coupled into a fiber input of the 50/50 coupler 316. Polarization states between reference and sample arms are matched through the use of fiber paddle polarization controllers 322, such as Thorlabs FPC030. The output light is then collimated with a collimator 320a, such as Thorlabs T06APC-1064. The other fiber output of the 80/20 coupler 318 is connected to the OCT sample arm. A tunable lens 330, which may be electronically or manually controlled, enables focusing to compensate for diopter variations between individuals. The tunable lens 330 may be electronically tunable, such as an Optotune EL-3-10, or manually focusable, such as an Optotune ML-20-37. An OCT 2D scanning motion compensating mirror 326 is placed conjugate to the retinal image plane to compensate for motion during OCT image acquisition. Offset galvo scanning mirrors 328 provide 2d scanning of the OCT beam to enable image formation. A pair of lenses or lens systems 310 are configured in a 4F imaging telescope configuration such that the offset galvo scanning mirrors are at an image conjugate of the ocular pupil and the second lens system is the second lens system of the optotype 4F imaging telescope. A dichroic mirror 412 is placed within the 4F imaging telescope to integrate optotype visual stimulus wavelengths. Finally, lens pair 110, here an N-FK51A Lens pair, functions as second lens pair of OCT imaging 4F telescope and second lens pair of optotype relay 4F telescope in the optical path to the retina 104 of eye 100. Iris camera illumination is provided by a ring of other configuration of LEDs 108a and 180b.

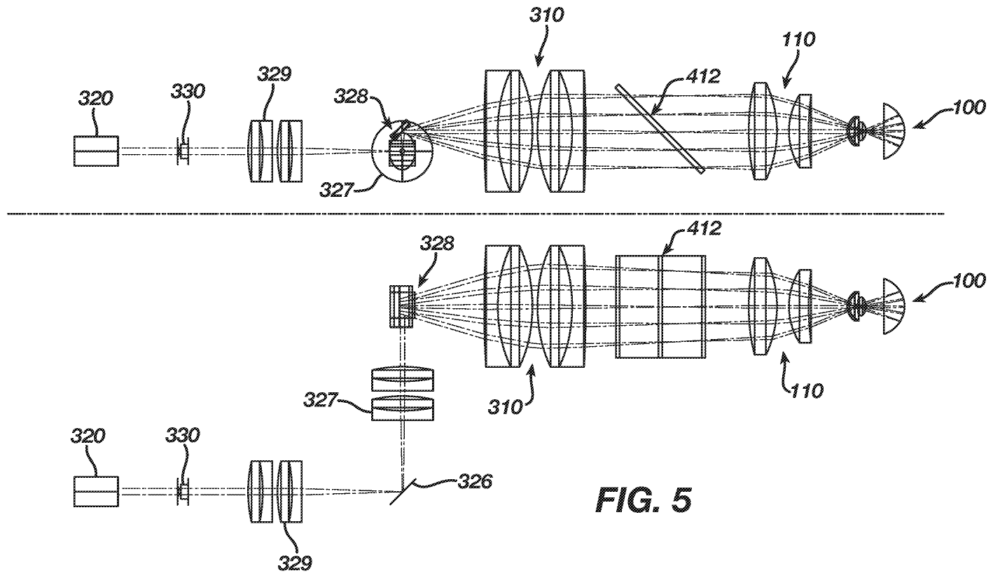

Turning now to FIG. 5, a ray diagram of an OCT imaging system portion of an exemplary ophthalmic system according to an embodiment is illustrated. The region at the top of the figure above the broken line represents the side view of the OCT imaging system whereas the lower portion of the figure below the broken line represents the top view of the OCT imaging system. An OCT sample arm collimator 320 such as Thorlabs TC06APC-1064 collimates an OCT light source. An electronically controllable tunable lens 330 such as Optotune EL-3-10-NIR provides OCT diopter focus control. The OCT beam next traverses an achromatic lens pair 329 such as Edmund Optics 45-806, which serves as a first lens pair of OCT 2D motion compensating mirror 4F telescope before entering OCT 2d motion compensating mirror 326, in this embodiment implemented using an Optotune MR-15 mirror-30 mirror. An achromatic lens pair 327 such as an Edmund Optics 45-805 functions as a second lens pair of OCT 2D motion compensating mirror 4F telescope. Retinal scanner 328 is conjugate to the ocular pupil with the OCT imaging 4f telescope. Lens pair 310 functions as the first lens pair of OCT imaging 4f telescope. Dichroic mirror 412, here a Semrock FF872-D101-42X50, integrates the OCT and optotype optical paths. Finally, lens pair 110, here an N-FK51A Lens pair functions as second lens pair of OCT imaging 4F telescope and second lens pair of optotype relay 4F telescope in the optical path to eye 100.

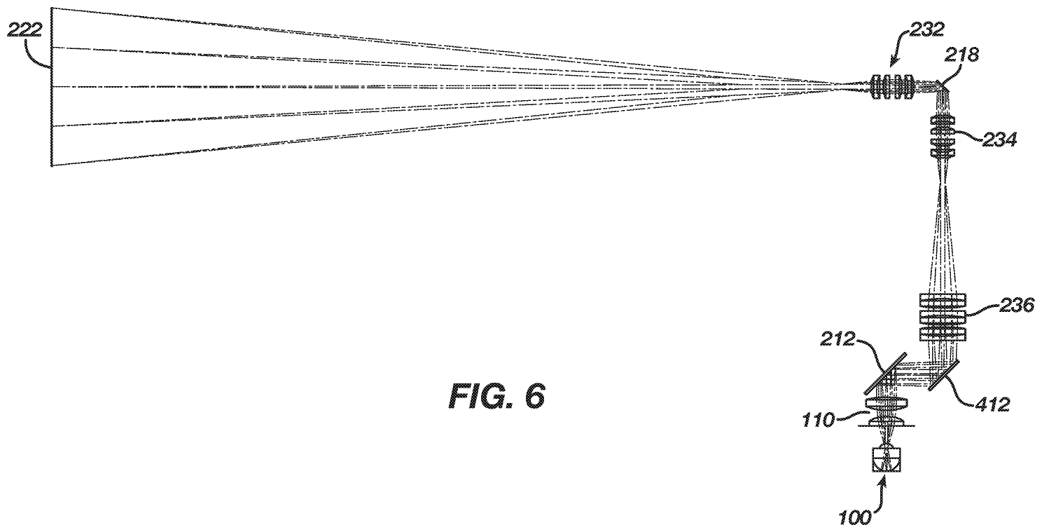

FIG. 6 illustrates a ray diagram of the optotype generator portion of the exemplary ophthalmic system corresponding to the FIG. 5. An optical stimulus is provided by OLED video screen 222 in this example a DFR0524 video screen optotype. A manual tunable lens implemented as Optotune, ML-20-37 with limiting aperture allows optotype Diopter focus control. An achromatic lens system, specifically two air spaced achromatic doublets combine to form a single lens system functioning as a first lens system of optotype 2D motion compensating mirror 4F telescope. A 2d motion compensating mirror 218 is implemented using an Optotune MR-15 mirror. An achromatic lens system, two air spaced achromatic doublets 232, form a single lens system that functions as a first lens of a 4F telescope. A second achromatic lens system, two air spaced achromatic doublets 234, forms a single lens system that functions as a second lens of optotype 2D motion compensating mirror 4F telescope. Achromatic triplet lens system (Edmund Optics 49-279) 236 functions as the first lens system of the optotype relay 4F telescope. A dichroic mirror 212 implemented using Edmund Optics 64-439 integrates the optotype and iris camera optical systems. Dichroic mirror 412, here a Semrock FF872-D101-42X50, integrates the OCT and optotype optical paths. Finally, an N-FK51A lens pair 110 functions as a second lens pair of the OCT imaging 4F telescope and a second lens pair of optotype relay 4F telescope along the beam path toward eye 100.

The foregoing description, for purposes of explanation, used specific nomenclature to provide a thorough understanding of the described embodiments. However, it will be apparent to one skilled in the art that many of the specific details are not required in order to practice the described embodiments. Thus, the foregoing descriptions of the specific embodiments described herein are presented for the purposes of illustration and description. They are not targeted to be exhaustive or to limit the embodiments to the precise forms disclosed. It will be apparent to one of ordinary skill in the art that many modifications and variations are possible in view of the above teachings.

The Detailed Description section is intended to be used to interpret the claims. The Summary and Abstract sections may set forth one or more but not all exemplary embodiments of the present invention as contemplated by the inventors, and thus, are not intended to limit the present invention and the appended claims in any way.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-describe

d exemplary embodiments but should be defined only in accordance with the following claims and their equivalents.

What is claimed is:

1. An ophthalmic system comprising:
an optotype generator configured to provide a direct optical stimulus to a retina of an eye, wherein an angle of incidence of the direct optical stimulus upon the retina may be adjusted; and
an optical coherence tomography (OCT) imaging system configured to generate OCT images of the retina of the eye in response to the direct optical stimulus.

2. The ophthalmic system of claim 1 further comprising a camera configured to capture a plurality of images of the eye.

3. The ophthalmic system of claim 1, wherein the OCT imaging system comprises a swept-frequency laser.

4. The ophthalmic system of claim 3, wherein the laser is centered between about 1020 and 1080 nm with a sweep rate of above about 100 kHz and a bandwidth of at least about nm.

5. The ophthalmic system of claim 1 further comprising a spectrally balanced interferometer topology.

6. The ophthalmic system of claim 1, wherein the direct optical stimulus produced by the optotype generator comprises three channels wherein each channel is composed of a primary color.

7. The ophthalmic system of claim 6, wherein the optotype generator further comprises at least one of: a selectively controllable shutter configured to control a projection of light among a plurality of primary colors; and a direct light source and a filter capable of producing the primary color via secondary filtration of the direct light source.

8. The ophthalmic system of claim 1 further comprising an integrated pupil tracking system configured to provide control of an entry position to the pupil.

9. The ophthalmic system of claim 1, wherein at least one of the optotype generator and the OCT imaging system comprises a Badal optometer configured to facilitate adjustment of a focus of the direct optical stimulus.

10. The ophthalmic system of claim 1, wherein the optotype generator is configured to modulate a frequency or intensity of the direct optical stimulus.

11. The ophthalmic system of 10, wherein the frequency or intensity of the direct optical stimulus is modulated via at least one of a chopper, acoustic optic, or an electro optic modulator.

12. The ophthalmic system of claim 1 further comprising an electrically controllable birefringent element.

13. The ophthalmic system of claim 12, wherein the electrically controllable birefringent element comprises a polarizer and waveplate.

14. A method comprising:
providing, via an optotype generator, a direct optical stimulus to a retina of an eye;
selectively adjusting the angle of incidence of the direct optical stimulus upon the retina; and
generating, via an optical coherence tomography (OCT) imaging system, a plurality of OCT images of the retina of the eye in response to the direct optical stimulus.

15. The method of claim 14 further comprising capturing via a camera a plurality of images of the eye.

16. The method of claim 14 wherein the direct optical stimulus produced by the optotype generator comprises three channels wherein each channel is composed of a primary color.

17. The method of claim 14 further comprising at least one of selectively controlling a shutter to control a projection of light among a plurality of primary colors; and filtering a direct light source capable to produce the primary color.

18. The method of claim 14 further comprising controlling, via an integrated pupil tracking system, an entry position to the pupil of the direct optical stimulus.

19. The method of claim 14 further comprising modulating a frequency or intensity of the direct optical stimulus.

20. The method of claim 19 wherein the frequency or intensity of the direct optical stimulus is modulated via at least one of a chopper, acoustic optic, or an electro optic modulator.
